(19) United States
(12) Patent Application Publication
(10) Pub. No.: US 2012/0157716 A1
(43) Pub. Date: Jun. 21, 2012

(54) Methods for Preparing Diaryl Disulfides

(75) Inventors: Teruo Umemoto, Westminster, CO (US); Rajendra P. Singh, Broomfield, CO (US)

(73) Assignee: UBE INDUSTRIES, LTD., Ube-shi (JP)

(21) Appl. No.: 13/162,839

(22) Filed: Jun. 17, 2011

Related U.S. Application Data
(60) Provisional application No. 61/362,647, filed on Jul. 8, 2010.

(51) Int. Cl. C07C 319/14

(52) U.S. Cl. 568/25

(57) ABSTRACT

New and useful methods for preparing bulky diaryl disulfides from a benzene derivative and sulfur halide are disclosed.

### TECHNICAL FIELD

The invention relates to methods for preparing diaryl disulfides. Diaryl disulfides are useful intermediates in the production of various fluorinating agents.

### BACKGROUND OF THE INVENTION

Bulky diaryl disulfides such as bis(4-tert-butyl-2,6-dimethylphenyl) disulfide and bis(2,4,6-triisopropylphenyl) disulfide are important intermediates for production of useful fluorinating agents and intermediates (U.S. Pat. Nos. 7,265,247 and 7,381,846; WO 2010/022001; J. Am. Chem. Soc. 2010, Vol. 132, pp. 18199-18205; and J. Org. Chem. 2011, Vol. 76, pp. 3112-3121).

Conventional production methods for bis(4-tert-butyl-2,6-dimethylphenyl) disulfide include: (1) bromination of 5-tert-butyl-1,3-dimethylbenzene with bromine, followed by treatment of the resulting 2-bromo-5-tert-butyl-1,3-dimethylbenzene with magnesium and then with sulfur (J. Chem. Soc. Perkin 1, 1978, 1090-1100); and (2) chlorosulfonylation of 5-tert-butyl-1,3-dimethylbenzene with chlorosulfonic acid, followed by treatment of the resulting 4-tert-butyl-2,6-dimethylbenzenesulfonyl chloride with 33% HBr in acetic acid in the presence of phenol or with zinc dust and titanium tetrachloride in tetrahydrofuran (U.S. Pat. Nos. 4,218,476 and 7,381,846). However, method (1) above is a multi-step low yield method. Method (2) is a two-step method in which the second step produces a large amount of byproducts that are brominated phenols and thus an additional procedure is required to remove the byproducts. Alternatively, the second step of Method (2) requires an excess amount of titanium tetachloride and a large amount of tetrahydrofuran as a solvent, and needs to control vigorous and exothermic reaction of titanium tetrachloride and tetrahydrofuran.

Conventional production methods for bis(2,4,6-triisopropylphenyl) disulfide include: chlorosulfonylation of 1,3,5-triisopropylbenzene with chlorosulfonic acid, followed by treatment of the resulting 2,4,6-triisopropylbenzenesulfonyl chloride with iodotrimethylsilane (J. Am. Chem. Soc. 1943, Vol. 65, pp. 2430-2441 and J. Org. Chem. 1982, Vol. 47, pp. 4806-4808). This production method is two-steps and requires an expensive reagent, iodotrimethylsilane.

Therefore, these conventional production methods are problematic in that they are prohibitive in cost, particularly on an industrial scale.

The present invention is directed toward overcoming one or more of the problems discussed above.

### SUMMARY OF THE INVENTION

The present invention provides a novel method for preparing a diaryl disulfide represented by formula (I):

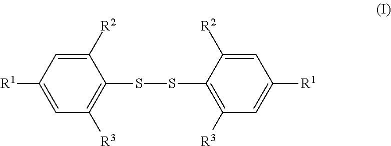

by reacting a benzene derivative represented by formula (II) with sulfur halide represented by formula S₂X₂, in the presence or absence of a Lewis acid:

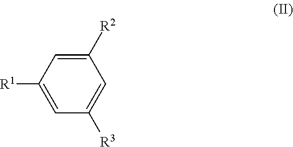

in which R¹, R², and R³ are independently a primary, secondary, or tertiary alkyl group having one to eight carbon atoms, wherein at least one of R¹, R², and R³ is a secondary or tertiary alkyl group having three to eight carbon atoms, and X is a halogen atom.

These and various other features and advantages of the invention will be apparent from a reading of the following detailed description and a review of the appended claims.

### DETAILED DESCRIPTION OF THE INVENTION

Embodiments of the present invention provide an industrially useful method for producing a diaryl disulfide represented by formula (I). Diaryl disulfides of formula (I) are useful intermediates for preparing arylsulfur trifluorides; arylsulfur trifluorides are useful fluorinating agents (U.S. Pat. Nos. 7,265,247 and 7,381,846; J. Am. Chem. Soc. 2010, Vol. 132, pp. 18199-18205; and J. Org. Chem. 2011, Vol. 76, pp. 3112-3121) and useful intermediates in their own right (WO 2010/022001). Unlike conventional methods in the art, the methods described herein are one-step and utilize inexpensive materials. The benzene derivatives are inexpensive and commercially available, and sulfur halides such as sulfur chloride (S₂Cl₂) are inexpensive and commercially available.

Embodiments of the invention include a method which comprises (see Scheme I) reacting a benzene derivative of formula (II) with a sulfur halide represented by formula S₂X₂, in the presence or absence of a Lewis acid.

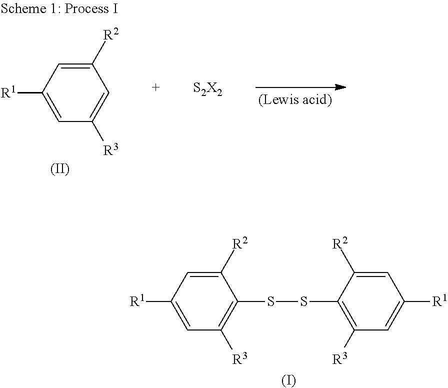

Table 1 provides chemical names and corresponding structures as well as their formula number for reference herein.

TABLE 1
Formulas I~II

| | | Compound |
|---|---|---|
| Chemical Name | Structure | number |
| Bis(4-tert-butyl-2,6- dimethylphenyl) disulfide | 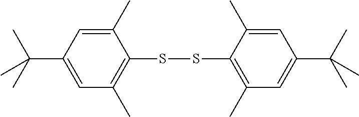 | Ia |
| Bis(2,4,6-triisopropylphenyl) disulfide | 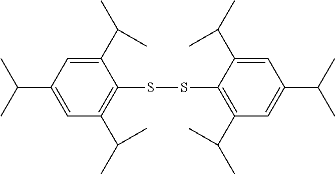 | Ib |
| 5-tert-Butyl-1,3-dimethylbenzene | 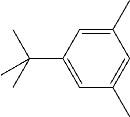 | IIa |
| 1,3,5-Triisopropylbenzene | 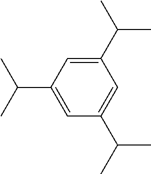 | IIb |

### Process I (Scheme 1)

Process I includes reacting compound (II) with a sulfur halide (S₂X₂) to form compound (I) in the presence or absence of a Lewis acid. Examples of sulfur halides are sulfur fluoride (S₂F₂), sulfur chloride (S₂Cl₂), sulfur bromide (S₂Br₂), and sulfur iodide (S₂I₂). Among them, S₂Cl₂ is most preferable from a view point of commercial availability and cost. S₂Cl₂ is industrially produced by reaction of Cl₂ and sulfur (see 8970 Sulfur chloride, The Merck Index, 14^{th} Ed., incorporated herein by reference for all purposes).

Benzene derivatives represented by formula (II) are commercially available or can be prepared in accordance with understood principles of synthetic chemistry. R¹, R², and R³ each is a primary, secondary, or tertiary alkyl group having one to eight carbon atoms, in which at least one of R¹, R², and R³ is a secondary or tertiary alkyl group having three to eight carbon atoms. Among them, R¹, R², and R³ each having one to four carbon atoms are preferable from a viewpoint of availability. Illustrative alkyl groups having one to four carbon atoms are methyl, ethyl, n-propyl, isopropyl, n-butyl, isobutyl, sec-butyl and tert-butyl. Methyl, ethyl, n-propyl, n-butyl, and isobutyl are primary alkyl groups; isopropyl and sec-butyl are secondary alkyl groups; and tert-butyl is a tertiary alkyl group. In some embodiments, a preferable secondary alkyl group is isopropyl and in some embodiments, a preferable tertiary alkyl group is tert-butyl. Illustrative benzene derivatives are 5-tert-butyl-1,3-dimethylbenzene, 5-tert-butyl-1,3-diethylbenzene, 5-tert-butyl-1,3-di(n-propyl)benzene, 5-tert-butyl-1,3-diisopropylbenzene, 5-isopropyl-1,3-dimethylbenzene, 5-isopropyl-1,3-diethylbenzene, 1,3-diisopropyl-5-methylbenzene, 1,3,5-triisopropylbenzene, and so on. Among them, in some aspects, 5-tert-butyl-1,3-dimethylbenzene and 1,3,5-triisopropylbenzene are preferable from a viewpoint of availability and usefulness.

The reaction can be conducted in the presence or absence of a Lewis acid. In one embodiment the reaction is conducted in the presence of a Lewis acid which allows the reaction to proceed at lower temperatures compared to the same reaction in the absence of a Lewis acid. Also, presence of the Lewis acid allows the reaction to proceed with a relatively shorter time frame compared to like reactions without a Lewis acid. Known Lewis acids can be used in the reaction of Process I. The known Lewis acids are exemplified by compounds or salts comprised of a cationic part of typical element or transition element and an anionic part of halide, carboxylate, oxide, sulfonate, sulfate, nitrate (NO₃), tetrafluoroborate (BF₄), and so on. The typical elements are exemplified by B, Al, Sn, P, As, Sb, and so on. The transition elements are exemplified by Zn, Cu, Fe, Ti, and so on. The halides are exemplified by fluoride, chloride, bromide, and iodide; the oxides by an oxygen atom(s); the carboxylates by formate, acetate, trifluoroacetate, propionate, oxalate, and so on; the sulfonates by methanesulfonate, trifluoromethanesulfonate, benzenesulfonate, toluenesulfonate, bromobenzenesulfonate, and so on; the sulfates by SO₄ and HSO₄. Hydrates of these Lewis acids can also be exemplified.

Transition element based Lewis acids are exemplified by zinc(II) salts or compounds such as ZnCl₂, ZnBr₂, ZnF₂, Znl₂, ZnO, Zn(OCOH)₂, Zn(OCOCH₃)₂, Zn(OCOC₂H₅)₂, Zn(OCOC₃H₇)₂, Zn(OCOCF₃)₂, Zn(OSO₂CH₃)₂, Zn(OSO₂Ph)₂, Zn(OSO₂C₆H₄CH₃)₂, Zn(OSO₂CF₃)₂, ZnSO₄, Zn(NO₃)₂, Zn(BF₄)₂, and so on, and iron(III) salts or compounds such as FeF₃, FeCl₃, FeBr₃, FeI₃, Fe₂O₃, and so on. Among them, zinc halide such as ZnCl₂, ZnBr₂, ZnF₂, and ZnI₂, and zinc carboxylate such as Zn(OCOH)₂, Zn(OCOCH₃)₂, Zn(OCOC₂H₅)₂, Zn(OCOC₃H₇)₂, and Zn(OCOCF₃)₂ can be desirable from a viewpoint of production yield and cost. Among zinc halides, zinc chloride, ZnCl₂, and zinc bromide, ZnBr₂, are more preferable and among zinc carboxylates, zinc acetate, Zn(OCOCH₃)₂, is more preferable because of cost.

Hydrates of these salts or compounds may also be used. Zn, Zn(I) salts or compounds, Fe, and Fe(II) salts or compounds can be used as these may be oxidized by sulfur halide represented by S₂X₂ to the corresponding Zn(II) and Fe(III) salts or compounds in the reaction mixture.

Reaction conditions of Process I can be optimized to obtain economically high yields of product, particularly where the reaction will be on an industrial scale. The amount of S₂X₂ can be selected in the range of about 0.5 mol or less, or about 0.5 mol to about 0.3 mol, against 1 mol of compound (II) to obtain a good yield of compound (I). The Lewis acid can act as a catalyst in this reaction. The amount of the Lewis acid can be selected from the range of a catalytic amount to a few equimolar amount, or about 0.1 mol % to about 50 mol %, or about 1 mol % to about 25 mol %, against the amount of S₂X₂.

In another embodiment, Process I is conducted in the presence of a solvent. Suitable solvents include carboxylic acids, acid anhydrides, hydrocarbons, halocarbons, nitro compounds, ethers, nitriles, carbon disulfide (CS₂), and so on. Illustrative carboxylic acids are formic acid, acetic acid, propionic acid, butyric acid, isobutyric acid, trifluoroacetic acid, and so on. Illustrative acid anhydrides are acetic anhydride, propionic anhydride, trifluoroacetic anhydride, and so on. Illustrative hydrocarbons include normal, branched, cyclic isomers of pentane, hexane, heptane, octane, nonane, decane, undecane, dodecane, and so on. Illustrative halocarbons include dichloromethane, chloroform, tetrachlorocarbon, dichloroethane, trichloroethane, tetrachloroethane, and so on. Illustrative nitro compounds are nitromethane, nitroethane, nitrobenzene, and so on. Illustrative ethers include diethyl ether, dipropyl ether, diisopropyl ether, dibutyl ether, diisobutyl ether, and so on. Illustrative nitriles include acetonitrile, propionitrile, and so on. Among these, in some embodiments, carboxylic acids are preferable, and among carboxylic acids, acetic acid can be more desirable from a viewpoint of production yield and cost.

In order to obtain relatively high yields of product in Process I, the reaction temperature can be selected in the range of about −30° C. to about +150° C., or about −15° C. to about +120° C., or about 0° C. to about +100° C., or about +10° C. to about +80° C.

Product compound (I) may be isolated by normal post-treatment procedures including filtration, extraction, precipitation, or crystallization.

According to the processes of the invention, bulky diaryl disulfides such as bis(4-tert-butyl-2,6-dimethylphenyl) disulfide and bis(2,4,6-triisopropylphenyl) disulfide are unexpectedly produced in high yields at low cost in comparison to conventional methodology.

The processes of the invention are particularly suited for commercial, large scale production. Relative to conventional methodologies used to produce diaryl disulfides, embodiments of the invention utilize relatively inexpensive chemicals, e.g., 5-tert-butyl-1,3-dimethylbenzene, S₂Cl₂, Lewis acids such as zinc salts and compounds, and solvents such as acetic acid, and require only one-step. The utilization of a one-step process allows for a simplified, less-costly, scale-up, fewer employees, less equipment, etc. The combination of these advantages provides unexpectedly large benefits in producing these important fluorinating agents (and intermediates).

### EXAMPLES

The following examples are provided for illustrative purposes only and are not intended to limit the scope of the invention.

### Example 1

### Preparation of bis(4-tert-butyl-2,6-dimethyphenyl) disulfide (Ia)

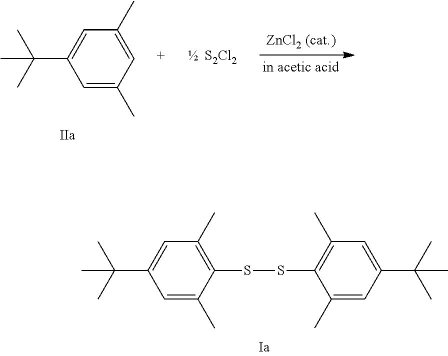

In a dry flask, 4-tert-butyl-2,6-dimethylbenzene (IIa) (34.39 g, 212 mmol), sulfur chloride (sulfur monochloride), S₂Cl₂, (14.31 g, 106 mmol), and 120 ml of glacial acetic acid were mixed. Anhydrous zinc chloride, ZnCl₂, (1.8 g, 13.3 mmol; 12.5 mol % against S₂Cl₂) was added into the mixture while stifling. Slight heat was generated with immediate evolution of hydrogen chloride gas, which was flowed into a sodium hydroxide solution with aid of nitrogen gas flow. The reaction mixture was stirred at room temperature for 4 h. Water (300 mL) was added to the reaction mixture and the mixture was stirred at room temperature for 10 minutes. The obtained solid was filtered, washed with water three times, and dried in air. The crude product was recrystallized from isopropanol to give 32.2 g (yield 79%) of bis(4-tert-butyl-2,6-dimethylphenyl) disulfide (Ia): M.p. 127-128° C.; ¹H NMR (CDCl₃) δ 7.03 (s, 4H, aromatic protons), 2.22 (s, 12H, 4×CH₃), 1.29 (s, 18H, 2×C(CH₃)₃); ¹³C NMR (CDCl₃) δ 152.7, 143.1, 131.9, 125.3, 34.7, 31.5, 22.0. Its purity was evaluated to be more than 98% by gas-layered chromatography. The reported melting point was 127-128° C. (see J. Chem. Soc. Perkin 1, 1978, pp. 1090-1100).

### Example 2

### Preparation of bis(4-tert-butyl-2,6-dimethyphenyl) disulfide (Ia)

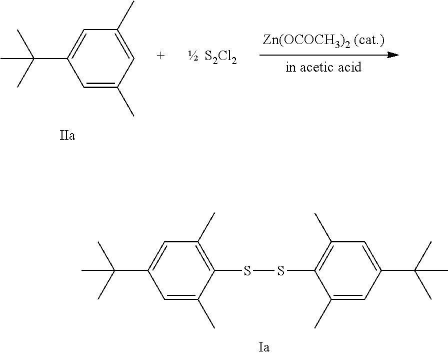

A dry one liter three-necked flask was set with a mechanical stirrer, internal thermometer, and nitrogen flow inlet, and outlet into a caustic scrubber solution. The flask was charged with 172.5 g (1.06 mol) of 5-tert-butyl-1,3-dimethylbenzene (IIa) and 300 mL of glacial acetic acid, followed by 43 mL (0.53 mol) of sulfur chloride, S₂Cl₂. The mixture was stirred for a few minutes under nitrogen flow at 20° C., and then 5.97 g (0.032 mol; 6 mol % against S₂Cl₂) of anhydrous zinc acetate, Zn(OCOCH₃)₂, was added to the mixture while stifling. The zinc acetate dissolved quickly and a rapid immediate reaction occurred with the evolution of hydrogen chloride gas, which was flowed into the scrubber solution with the aid of a flow of nitrogen gas. The reaction temperature rose to 37° C. over 2-3 minutes. The flask was cooled in a water bath so that the reaction temperature stayed below 40° C. The reaction mixture gradually became an orange slurry. After 3 hours, the water bath was removed and the reaction mixture was allowed to stir overnight without cooling. Total reaction time was 22 hrs. The slurry reaction mixture was quenched by the addition of 500 mL of water and stirred for a few minutes. The resulting precipitate was filtered, washed with an additional 500 mL of water, and dried in the air. The crude product obtained was recrystallized from isopropanol to give 161.3 g (79% yield) of bis(4-tert-butyl-2,6-dimethylphenyl) disulfide (Ia). Its purity was evaluated to be more than 99% by gas-layered chromatography. Physical and spectral data of the product are shown in Example 1.

### Example 3

### Preparation of bis(4-tert-butyl-2,6-dimethyphenyl) disulfide (Ia)

The reaction of Example 3 was conducted in the same way as in Example 2 except that 1.97 g (0.011 mol; 2 mol % against S₂Cl₂) of anhydrous zinc acetate, Zn(OCOCH₃)₂, was used in place of 5.97 g (0.032 mol; 6 mol % against S₂Cl₂) of anhydrous zinc acetate and the total reaction time was 30 hrs.

The yield of product (Ia) was 160.4 g (78%), and the purity of the product was evaluated to be more than 99% by gas-layered chromatography.

### Example 4

### Preparation of bis(4-tert-butyl-2,6-dimethyphenyl) disulfide (Ia)

The reaction of Example 4 was conducted in the same way as in Example 2 except that 1.44 g (0.011 mol; 2 mol % against S₂Cl₂) of anhydrous zinc chloride, ZnCl₂, was used in place of 5.97 g (0.032 mol; 6 mol % against S₂Cl₂) of anhydrous zinc acetate and the total reaction time was 24 hrs.

The yield of product (Ia) was 159.7 g (78%), and the purity of the product was evaluated to be more than 99% by gas-layered chromatography.

### Example 5

### Preparation of bis(2,4,6-triisopropylphenyl) disulfide (Ib)

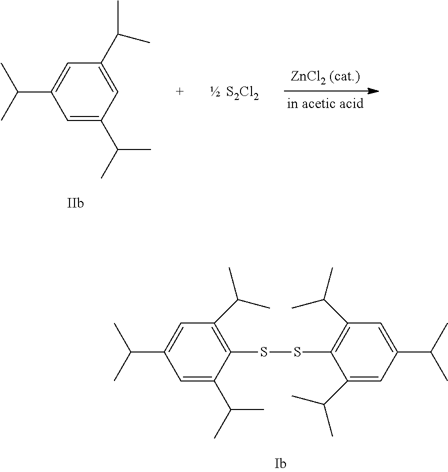

In a dry flask, 1,3,5-triisopropylbenzene (IIb) (43.25 g, 212 mmol), sulfur chloride, S₂Cl₂, (14.31 g, 106 mmol), and 120 ml of glacial acetic acid were mixed. Anhydrous zinc chloride, ZnCl₂, (4.0 g, 29.4 mmol; 28 mol % against S₂Cl₂) was added to the reaction mixture while stifling. The reaction mixture was stirred at room temperature for 1 h and then heated to 60° C. for 24 h. Hydrogen chloride gas generated was flowed into a sodium hydroxide solution along with a flow of nitrogen gas. After cooling, water (400 ml) was added to the reaction mixture and the mixture was stirred at room temperature for 10 minutes. The obtained solid was filtered, washed with water three times, and dried in air. The crude product was recrystallized from isopropanol/methanol to give 34.9 g (yield 70%) of bis(2,4,6-triisopropylphenyl) disulfide (Ib): M.p. 93-94° C.; ¹H NMR (CDCl₃) δ 6.92 (s, 4H, aromatic protons), 3.54 (septet, J=6.9 Hz, 4H, 4×CH), 2.83 (septet, J=6.9 Hz, 2H, 2×CH), 1.20 (d, J=6.9 Hz, 12H, 4×CH₃), 1.00 (d, J=6.9 Hz, 24H, 8×CH₃); ¹³C NMR (CDCl₃) δ 153.5, 150.6, 129.4, 121.9, 34.7, 31.5, 24.0 (br.). Its purity was evaluated to be more than 99% by gas-layered chromatography.

### Comparative Example 1

### Reaction of 1,3,5-trimethylbenzene with S₂Cl₂ in the presence of ZnCl₂ as a catalyst

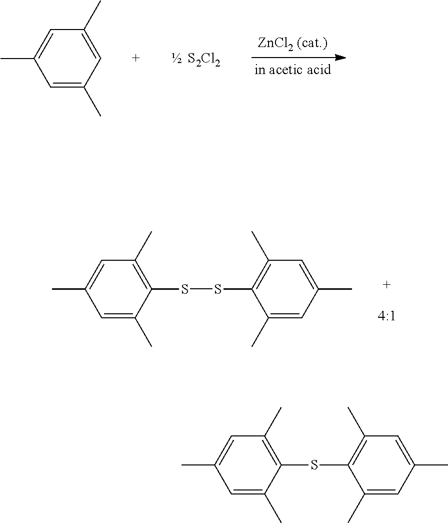

In a dry flask, 2,4,6-trimethylbenzene (48.0 g, 400 mmol), sulfur chloride, S₂Cl₂, (27 g, 200 mmol), and 120 ml of glacial acetic acid were mixed. Anhydrous zinc chloride, ZnCl₂, (5.0 g, 36.8 mmol; 18 mol % against S₂Cl₂) was added to the mixture while stirring. Heat was generated with immediate evolution of hydrogen chloride gas, which was flowed into a sodium hydroxide solution along with a flow of nitrogen gas. The reaction mixture was stirred at room temperature for 4 h. Water (500 mL) was added to the reaction mixture and the mixture was stirred for 10 minutes. The obtained solid was filtered, washed with water 3 times, and dried in air. The crude product was recrystallized from isopropanol/methanol to give 48.3 g of products, which were evaluated to be a 4:1 mixture of bis(2,4,6-trimethylphenyl) disulfide and bis(2,4,6-trimethylphenyl) sulfide by gas-layered chromatography (GC) and GC-Mass spectral analysis.

From Comparative Example 1 above, it was shown that 1,3,5-trimethylbenzene, a case of R¹═R²═R³=a primary alkyl group of formula (II), provides a 4:1 mixture of diaryl di- and mono-sulfide which are difficult to separate. It has also been known that 1,3,5-trimethylbenzene reacts with S₂Cl₂ in ether to produce a large amount (42%) of bis(1,3,5-trimethylphenyl) tetrasulfide in addition to bis(1,3,5-trimethylphenyl) disulfide (19%) (see J. Chem. Soc. 1961, pp. 4510-4514).

The present invention provides a method of exclusively producing (or nearly exclusively producing) high yields of diaryl disulfides. Therefore, the present invention is based on this unexpected finding with respect to the bulky benzene derivatives represented by formula (II) in which at least one of R¹, R², and R³ is a secondary or tertiary alkyl group.

All references including publications and patents are incorporated by reference herein for all purposes.

What is claimed is:

1. A method for preparing a diaryl disulfide represented by formula (I): 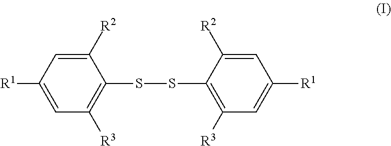
the method comprising: reacting a benzene derivative represented by formula (II), 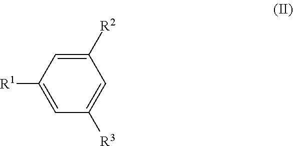
with a sulfur halide represented by S₂X₂;
in which R¹, R², and R³ are independently a primary, secondary, or tertiary alkyl group having one to eight carbon atoms, wherein at least one of R¹, R², and R³ is a secondary or tertiary alkyl group having three to eight carbon atoms, and X is a halogen atom.

2. The method of claim 1, wherein X is a chlorine atom.

3. The method of claim 1, wherein R¹, R², and R³ each has one to four carbons.

4. The method of claim 1, wherein the secondary alkyl group is an isopropyl group.

5. The method of claim 1, wherein the tertiary alkyl group is a tert-butyl group.

6. The method of claim 1, further comprising a Lewis acid.

7. The method of claim 6, wherein the Lewis acid is a zinc salt or compound.

8. The method of claim 7, wherein zinc salt or compound is a zinc halide.

9. The method of claim 7, wherein zinc salt or compound is a zinc carboxylate.

10. The method of claim 7, wherein zinc salt or compound is zinc acetate.

11. The method of claim 1, wherein the step of reacting is performed in the presence of a solvent.

12. The method of claim 11, wherein the solvent is a carboxylic acid.

13. The method of claim 11, wherein the solvent is acetic acid.

14. The method of claim 1, wherein the reaction is performed at a temperature in a range of about 0° C. to about +100° C.

15. The method of claim 1, wherein the benzene derivative is a compound selected from the group consisting of 5-tert-butyl-1,3-dimethylbenzene and 1,3,5-triisopropylbenzene.
